(19) United States
(12) Patent Application Publication
(10) Pub. No.: US 2021/0052676 A1
(43) Pub. Date: Feb. 25, 2021

(54) ANTI-OXIDANT COMPOSITION WITH LACTIC ACID BACTERIUM STRAIN OR FERMENTATION METABOLITE THEREOF AND USES THEREOF

(71) Applicant: GLAC BIOTECH CO., LTD., Tainan City (TW)

(72) Inventors: PEI-SHAN HSIEH, Tainan City (TW); CHUNG-WEI KUO, Tainan City (TW); YI-CHUN TSAI, Tainan City (TW); HSIEH-HSUN HO, Tainan City (TW); YI WEI KUO, Tainan City (TW); JIA-HUNG LIN, Tainan City (TW)

(21) Appl. No.: 16/940,886

(22) Filed: Jul. 28, 2020

(30) Foreign Application Priority Data
Aug. 22, 2019 (CN) 201910780776.2
Nov. 22, 2019 (TW) 108142643

(51) Int. Cl. A61K 35/747; A61K 35/745; A61K 9/00; A61K 8/99

(52) U.S. Cl. CPC A61K 35/747; A61K 35/745; A61K 8/99; A61K 9/0056; A61K 9/0014

(57) ABSTRACT

An isolated lactic acid bacterium strain or fermentation metabolite thereof has an anti-oxidant function and is in form of a food composition or a pharmaceutical composition, wherein the isolated lactic acid bacterium strain comprises at least one of an OLP-01 strain of Bifidobacterium longum subsp. longum; a Bv-889 strain of Bifidobacterium breve; a BLI-02 strain of Bifidobacterium longum subsp. infantis; a CP-9 strain of Bifidobacterium animalis subsp. lactis; a Bf-688 strain of Bifidobacterium bifidum; a GL-104 strain of Lactobacillus reuteri; an AP-32 strain of Lactobacillus salivarius subsp. salicinius; and a bv-77 strain of Lactobacillus rhamnosus.

### FIELD OF THE INVENTION

The present invention relates to a composition and uses thereof, particularly to an anti-oxidant composition with a lactic acid bacterium strain or a fermentation metabolite thereof and uses thereof.

### DESCRIPTION OF THE PRIOR ART

Aging is the main factor degrading human organs. Aging of organs may cause chronic kidney diseases, dementia, cardiovascular diseases, diabetes, cancers, and other chronic diseases, and may even lead to death of a person. Free radicals generated by human bodies is the main cause leading to aging of organs. Free radicals generated by human bodies may be classified into reactive oxygen species and reactive nitrogen species. Reactive oxygen species are byproducts of normal metabolism in organisms, including oxygen ions and hydrogen peroxide. The reactive oxygen materials play very important roles in conducting cellular signals, resisting microbe infection, and maintaining organism constancy. While pathogenic bacteria invade an organism, reactive nitrogen materials are massively generated by immune cells to kill the pathogenic bacteria. Vascular endothelial cells also secrete some reactive nitrogen materials to facilitate vasodilation and signal transduction. Therefore, regulating and maintaining the balance of free radicals is critical to health.

While the mechanism of regulating free radicals fails to work, excessive free radicals will damage DNA, vary the structures of intracellular proteins, attack cell membranes, and even cause the death of cells. The excessive reactive oxygen materials, which are generated by chronic inflammation and accumulated by the kidneys, is one of the factors causing chronic kidney diseases. The excessive reactive oxygen materials may kill kidney cells. Thus, the function of kidneys will be gradually degraded, and the patients can only survive on blood dialysis in the end stage. Amyloid accumulated inside the brain induces chronic inflammation. Free radicals generated by chronic inflammation may lead to the death of neurons and finally bring about the Alzheimer's disease (dementia). Long-time UV exposure or heat exposure causes reactive oxygen materials to dramatically increase. The reactive oxygen materials may cause skin aging, skin darkening or even lead to skin cancers.

Accordingly, it is an urgency to develop a nutrient, which can be used long term and has an anti-oxidant function. In general, lactic acid bacteria are safe for human beings. Therefore, finding out lactic acid bacterium strains having a function of anti-oxidation and the fermentation metabolites thereof becomes a target the manufacturers are eager to achieve.

### SUMMARY OF THE INVENTION

The present invention provides a composition with a lactic acid bacterium strain or a fermentation metabolite thereof, which has an anti-oxidant effect, and which is able to reduce concentration of free radicals and inhibit aging of organs.

In one embodiment, the anti-oxidant composition with a lactic acid bacterium strain or a fermentation metabolite thereof of the present invention comprises an isolated lactic acid bacterium strain having an active anti-oxidant effect or a fermentation metabolite thereof; and an excipient, diluent or carrier. The lactic acid bacterium strain includes at least one of an OLP-01 strain of Bifidobacterium longum subsp. longum (CGMCC No. 17345); a Bv-889 strain of Bifidobacterium breve (CGMCC No. 16145); a BLI-02 strain of Bifidobacterium longum subsp. infantis (CGMCC No. 15212); a CP-9 strain of Bifidobacterium animalis subsp. lactis (CCTCC NO: M2014588); a Bf-688 strain of Bifidobacterium bifidum (CGMCC No. 17953); a GL-104 strain of Lactobacillus reuteri (CCTCC NO: M209138); an AP-32 strain of Lactobacillus salivarius subsp. salicinius (CCTCC NO: M2011127); and a bv-77 strain of Lactobacillus rhamnosus (CCTCC NO: M2014589).

In another embodiment, the present invention proposes a use of a composition with a lactic acid bacterium strain or a fermentation metabolite thereof for anti-oxidation comprising administering to a subject the composition, wherein the composition with the lactic acid bacterium strain or the fermentation metabolite thereof comprises an isolated lactic acid bacterium strain having an active anti-oxidant effect or a fermentation metabolite thereof; and an excipient, diluent or carrier. The lactic acid bacterium strain includes at least one of an OLP-01 strain of Bifidobacterium longum subsp. longum (CGMCC No. 17345); a Bv-889 strain of Bifidobacterium breve (CGMCC No. 16145); a BLI-02 strain of Bifidobacterium longum subsp. infantis (CGMCC No. 15212); a CP-9 strain of Bifidobacterium animalis subsp. lactis (CCTCC NO: M2014588); a Bf-688 strain of Bifidobacterium bifidum (CGMCC No. 17953); a GL-104 strain of Lactobacillus reuteri (CCTCC NO: M209138); an AP-32 strain of Lactobacillus salivarius subsp. salicinius (CCTCC NO: M2011127); and a bv-77 strain of Lactobacillus rhamnosus (CCTCC NO: M2014589). The abovementioned strains are respectively deposited in China General Microbiological Culture Collection Center (CGMCC) and China Center for Type Culture Collection (CCTCC).

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1 shows the results of the experiments for determining the free radical eliminating ability of the lactic acid bacterium strains of the present invention;

FIG. 2 shows the results of the experiments for determining the free radical eliminating ability of the fermentation metabolites of the lactic acid bacterium strains of the present invention;

FIG. 3 shows the results of the experiments to determine the reduction ability of the lactic acid bacterium strains of the present invention;

FIG. 4 shows the results of the experiments to determine the reduction ability of the fermentation metabolites of the lactic acid bacterium strains of the present invention;

FIG. 5 shows the results of analyzing the activity of the lactic acid bacterium strains of the present invention to induce the intestinal epithelial cells to generate superoxide dismutase;

FIG. 6 shows the results of analyzing the activity of the lactic acid bacterium strains of the present invention to induce the intestinal epithelial cells to generate catalase; and

FIG. 7 shows the results of analyzing the activity of 10% aqueous solutions of the powders of the fermentation metabolites of the lactic acid bacterium strains of the present invention to induce the intestinal epithelial cells to generate superoxide dismutase.

### DESCRIPTION OF THE PREFERRED EMBODIMENTS

Various embodiments of the present invention will be described in detail below and illustrated in conjunction with the accompanying drawings. In addition to these detailed descriptions, the present invention can be widely implemented in other embodiments, and apparent alternations, modifications and equivalent changes of any mentioned embodiments are all included within the scope of the present invention and based on the scope of the Claims. In the descriptions of the specification, in order to make readers have a more complete understanding about the present invention, many specific details are provided; however, the present invention may be implemented without parts of or all the specific details. In addition, the well-known steps or elements are not described in detail, in order to avoid unnecessary limitations to the present invention. Same or similar elements in Figures will be indicated by same or similar reference numbers. It is noted that the Figures are schematic and may not represent the actual size or number of the elements. For clearness of the Figures, some details may not be fully depicted.

The freeze-dried cultures of the lactic acid bacterium strains mentioned in the specification are deposited in China General Microbiological Culture Collection Center (CGMCC) of Chinese Academy of Sciences (NO. 1 West Beichen Road, Chaoyang District, Beijing 100101, China)) and China Center for Type Culture Collection (CCTCC) of Wuhan University (Wuhan 430072, China). The details thereof are listed in Table. 1.

TABLE 1
Data of Deposited Lactic Acid Bacterium Strain

| Strain | Specie | Deposition No. | Deposition Date |
|---|---|---|---|
| OLP-01 | Bifidobacterium | CGMCC No. 17345 | Mar. 18, 2019 |
| | longum subsp. longum | | |
| Bv-889 | Bifidobacterium breve | CGMCC No. 16145 | Jul. 23, 2018 |
| BLI-02 | Bifidobacterium longum | CGMCC No. 15212 | Jan. 15, 2018 |
| | subsp. infantis | | |
| CP-9 | Bifidobacterium animalis | CCTCC NO: M2014588 | Nov. 24, 2014 |
| | subsp. lactis | | |
| Bf-688 | Bifidobacterium bifidum | CGMCC No. 17953 | Jun. 18, 2019 |
| GL-104 | Lactobacillus reuteri | CCTCC NO: M209138 | Aug. 7, 2009 |
| AP-32 | Lactobacillus salivarius | CCTCC NO: M2011127 | Apr. 10, 2011 |
| | subsp. salicinius | | |
| bv-77 | Lactobacillus rhamnosus | CCTCC NO: M2014589 | Nov. 24, 2014 |

It is found: the deposited strains listed in Table.1 and the fermentation metabolites thereof have an active effect of anti-oxidation and removing free radicals, including the OLP-01 strain of Bifidobacterium longum subsp. longum; the Bv-889 strain of Bifidobacterium breve; the BLI-02 strain of Bifidobacterium longum subsp. Infantis; a CP-9 strain of Bifidobacterium animalis subsp. lactis; a Bf-688 strain of Bifidobacterium bifidum; a GL-104 strain of Lactobacillus reuteri; an AP-32 strain of Lactobacillus salivarius subsp. salicinius; and a bv-77 strain of Lactobacillus rhamnosus. Therefore, the deposited strains listed in Table.1 and the fermentation metabolites thereof may be used in anti-oxidation and removing free radicals.

In one embodiment, the composition with a lactic acid bacterium strain or a fermentation metabolite thereof of the present invention, which is used in anti-oxidation and removing free radicals, comprises a lactic acid bacterium strain or a fermentation metabolite thereof; and an excipient, diluent or carrier. The lactic acid bacterium strain is at least one isolated lactic acid bacterium strain selected from a group including a OLP-01 strain of Bifidobacterium longum subsp. longum (CGMCC No. 17345); a Bv-889 strain of Bifidobacterium breve (CGMCC No. 16145); a BLI-02 strain of Bifidobacterium longum subsp. infantis (CGMCC No. 15212); a CP-9 strain of Bifidobacterium animalis subsp. lactis (CCTCC NO: M2014588); a Bf-688 strain of Bifidobacterium bifidum (CGMCC No. 17953); a GL-104 strain of Lactobacillus reuteri (CCTCC NO: M209138); an AP-32 strain of Lactobacillus salivarius subsp. salicinius (CCTCC NO: M2011127); and a bv-77 strain of Lactobacillus rhamnosus (CCTCC NO: M2014589). The abovementioned strains are respectively deposited in China General Microbiological Culture Collection Center (CGMCC) and China Center for Type Culture Collection (CCTCC). In one embodiment, the excipient, diluent or carrier is a physiologically-acceptable excipient, diluent or carrier; thus, the composition with a lactic acid bacterium strain or a fermentation metabolite thereof of the present invention may be used as a food composition. In one embodiment, the excipient, diluent or carrier is a pharmaceutically-acceptable excipient, diluent or carrier; thus, the composition with a lactic acid bacterium strain or a fermentation metabolite thereof of the present invention may be used as a pharmaceutical composition. Alternatively, the excipient, diluent or carrier is a cosmeceutically-acceptable excipient, diluent or carrier; thus, the composition with a lactic acid bacterium strain or a fermentation metabolite thereof of the present invention may be used as a cosmetic composition.

In the embodiment of a food composition, the physiologically-acceptable excipient, diluent or carrier may be a food. The food may be but is not limited to be dairy food, tea, coffee, a chewing gum, a tooth-cleaning candy (such as an oral strip, a chewable tablet, or jelly sweets), or a combination thereof. The dairy food may be fermented milk, yoghurt, cheese, milk drink, or powdered milk. In the embodiment of a pharmaceutical composition, the pharmaceutical composition may be in form of an oral dosage or a topical dosage. For example, the oral dosage may be in form of a tablet, a capsule, a solution, or a powder.

In the embodiment of a cosmetic composition, the cosmeceutically-acceptable excipient, diluent or carrier may be: 1) liquid cosmetics, such as shower gel, shampoo, lotion, perfume, etc.; 2) emulsion cosmetics; 3) cream cosmetics, such as moisturizing cream, foundation cream, paste shampoo; 4) powder cosmetics, such as fragrance powder, talcum powder; 5) block cosmetics, such as pressed powder, cosmetic box; 6) stick cosmetics, such as lipstick, hair wax.

In the embodiment of the composition with a lactic acid bacterium strain, the number of the lactic acid bacterium strains may be over 10⁶ CFU (Colony-Forming Unit), preferably over 10¹⁰ CFU. In the embodiment of the composition with a fermentation metabolite of a lactic acid bacterium, the fermentation metabolite may contain a deactivated strain, a fermentation liquid where bacteria are removed, or a dried powder of a fermentation liquid where bacteria are removed. In one embodiment, the fermentation liquid is a supernatant of fermentation, or a fermentation whey. In one embodiment, the fermentation metabolite of a lactic acid bacterium contains more than 0.5% of the dried powder of the fermentation metabolite or more than 2.5% of the fermentation liquid of the fermentation metabolite.

### Embodiment I: Morphology and General Properties of the Strains of the Present Invention

The taxonomic characteristics of the strain are identified with the 16S rDNA sequencing analysis and the API bacterial identification system. The morphology and general properties of the strains are listed in Table.2.

TABLE 2
Morphology and General Properties of Lactic Acid
Bacterium Strain of the Present Invention

| Strain | Morphology and Characteristics | |
|---|---|---|
| OLP-01 of | 1. | They are gram-positive bacilli, unlikely to generate spores, free of |
| Bifidobacterium | | catalase, oxidase and motility, able to grow in obligately-anaerobic |
| longum subsp. | | environments, most suitable to grow at a temperature of 37 ± 1° C. |
| longum | | They belong to facultative heterofermentative strains and do not |
| | | generate gas in glucose metabolism. |
| | 2. | The colonies grown in MRS agar are in form of solid circles in white |
| | | color. The bacterium body has a middle-size or longer rod-like |
| | | shape, and two ends thereof sometimes have Y or V-shaped |
| | | branches. |
| Bv-889 of | 1. | They are gram-positive bacilli, unlikely to generate spores, free of |
| Bifidobacterium | | catalase, oxidase and motility, able to grow in obligately-anaerobic |
| breve | | environments, most suitable to grow at a temperature of 37 ± 1° C. |
| | | They belong to facultative heterofermentative strains and do not |
| | | generate gas in glucose metabolism. |
| | 2. | The colonies grown in MRS agar are in form of solid circles in white |
| | | color. The bacterium body has a middle-size or shorter rod-like |
| | | shape, and two ends thereof sometimes have Y or V-shaped |
| | | branches. |
| BLI-02 of | 1. | They are gram-positive bacilli, unlikely to generate spores, free of |
| Bifidobacterium | | catalase, oxidase and motility, able to grow in obligately-anaerobic |
| longum subsp. | | environments, most suitable to grow at a temperature of 37 ± 1° C. |
| infantis | | They belong to facultative heterofermentative strains and do not |
| | | generate gas in glucose metabolism. |
| | 2. | The colonies grown in MRS agar are in form of solid circles in white |
| | | color. The bacterium body has a middle-size or longer rod-like |
| | | shape, and two ends thereof sometimes have Y or V-shaped |
| | | branches. |
| CP-9 of | 1. | They are gram-positive bacilli, unlikely to generate spores, free of |
| Bifidobacterium | | catalase, oxidase and motility, able to grow in obligately-anaerobic |
| animalis subsp. | | environments, most suitable to grow at a temperature of 37 ± 1° C. |
| lactis | | They belong to facultative heterofermentative strains and do not |
| | | generate gas in glucose metabolism. |
| | 2. | The colonies grown in MRS agar are in form of solid circles in white |
| | | color. The bacterium body has a middle-size or longer rod-like |
| | | shape, and two ends thereof sometimes have Y or V-shaped |
| | | branches. |
| Bf-688 of | 1. | They are gram-positive bacilli, unlikely to generate spores, free of |
| Bifidobacterium | | catalase, oxidase and motility, able to grow in obligately-anaerobic |
| bifidum | | environments, most suitable to grow at a temperature of 37 ± 1° C. |
| | | They belong to facultative heterofermentative strains and do not |
| | | generate gas in glucose metabolism. |
| | 2. | The colonies grown in MRS agar are in form of solid circles in white |
| | | color. The bacterium body has a middle-size or longer rod-like |
| | | shape, and two ends thereof sometimes have Y or V-shaped |
| | | branches. |
| GL-104 of | 1. | The colonies grown in MRS agar are in form of solid circles in white |
| Lactobacillus | | color. The bodies of the bacteria each have a shape of a short rod, |
| reuteri | | and the ends of the body are circular-shaped. They often appear in |
| | | single bodies. |
| | 2. | They are gram-positive bacilli, unlikely to generate spores, free of |
| | | catalase, oxidase and motility, able to grow in aerobic and anaerobic |
| | | environments, most suitable to grow at a temperature of 37 ± 1° C. |
| | | They belong to facultative heterofermentative strains and do not |
| | | generate gas in glucose metabolism. |
| AP-32 of | 1. | They are gram-positive bacilli, unlikely to generate spores, free of |
| Lactobacillus | | catalase, oxidase and motility, able to grow in aerobic and anaerobic |
| salivarius subsp. | | environments, most suitable to grow at a temperature of 37 ± 1° C. |
| salicinius | | They belong to facultative heterofermentative strains and do not |
| | | generate gas in glucose metabolism. |
| | 2. | The colonies grown in MRS agar are in form of solid circles in white |
| | | color. The bodies of the bacteria each have a shape of a short rod, |
| | | and the ends of the body are circular-shaped. They often appear in |
| | | single bodies. |
| bv-77 of | 1. | They are gram-positive bacilli, unlikely to generate spores, free of |
| Lactobacillus | | catalase, oxidase and motility, able to grow in aerobic and anaerobic |
| rhamnosus | | environments, most suitable to grow at a temperature of 37 ± 1° C. |
| | | They belong to facultative heterofermentative strains and do not |
| | | generate gas in glucose metabolism. |
| | 2. | The colonies grown in MRS agar are in form of solid circles in white |
| | | color. The bodies of the bacteria each have a shape of a short rod, |
| | | and the ends of the body are circular-shaped. They often appear in |
| | | single bodies. |

### Embodiment II: Collection, Cultivation and Preservation of the Lactic Acid Bacterium Strains of the Present Invention

The strain of the present invention is preserved in 20% glycerol at a temperature of −80° C. Before use, the strain is activated twice with MRS broth (DIFCO) containing 0.05% cysteine at a temperature of 37° C. for 24 hours. In the present invention, the OLP-01 strain of Bifidobacterium longum subsp. Longum and the GL-104 strain of Lactobacillus reuteri are separated from human intestines; the Bv-889 strain of Bifidobacterium breve, the BLI-02 strain of Bifidobacterium longum subsp. Infantis, the CP-9 strain of Bifidobacterium animalis subsp. Lactis and the Bf-688 strain of Bifidobacterium bifidum are separated from human breast milk; the AP-32 strain of Lactobacillus salivarius subsp. salicinius is separated from human excrement. The fermentation metabolite of the present invention is the fermentation product generated by at least one of the abovementioned lactic acid bacterium strains. The fermentation product is centrifuged, filtered, sterilized and then purified to obtain a fermentation liquid. According to requirement, the fermentation liquid may be further dried to form fermentation powder of the lactic acid bacterium. The fermentation liquid or fermentation powder can be stored at an ambient temperature.

### Embodiment III: Analysis of the Free Radical Eliminating Ability of the Lactic Acid Bacterium Strains

DPPH (di(phenyl)-(2,4,6-trinitrophenyl) iminoazanium) is a stable free-radical molecule. DPPH free radicals in methanol have a maximum absorbance at a wavelength of 517 nm. While DPPH free radicals react with anti-oxidation materials, the anti-oxidation materials provide hydrogen ions (protons) to eliminate the free radicals. Thus, the ianthinus color of the DPPH free radicals decays, and the absorbance at 517 nm decreases. The value of OD₅₁₇ is used to determine the free radical eliminating ability of the tested lactic acid bacterium strains.

The method to determine the free radical eliminating ability of the lactic acid bacterium strains is introduced as follows. By a ratio of 1:1, respectively mix 0.2 mM DPPH methanol solutions homogeneously with the following liquids: the suspensions of the lactic acid bacterium strains of the present invention at a concentration of about 6×10⁹ CFU; the suspensions of the reference lactic acid bacterium strains, including a GL-156 strain of Lactobacillus paracasei, a TYCA06 strain of Lactobacillus acidophilus, an MH-68 strain of Lactobacillus johnsonii and an F-1 strain of Lactobacillus rhamnosus at a concentration of about 6×10⁹ CFU; a 2.5 μg/ml Vitamin C solution (used as a positive control group); a liquid containing an SY-66 strain (free of anti-oxidant activity) of Streptococcus thermophiles (used as a negative control group); and double-distilled water (used as a blank group). Next, let the mixture liquids react in the dark at an ambient temperature for 30 minutes. Next, the mixture liquids are centrifuged at a speed of 12000 rpm at a temperature of 4° C. for 2 minutes. Next, take 200 μl of liquid from the mixture liquids to a 96-well plate, and measure the values of OD₅₁₇ thereof. The equation for calculating the free radical eliminating ability is expressed as

Free Radical Eliminating Ability=OD_{blank}−ODₛₐₘₚₗₑ/OD_{blank}*100%,

wherein ODₛₐₘₚₗₑ is the absorbance of the tested sample and OD_{Blank} is the absorbance of the blank group.

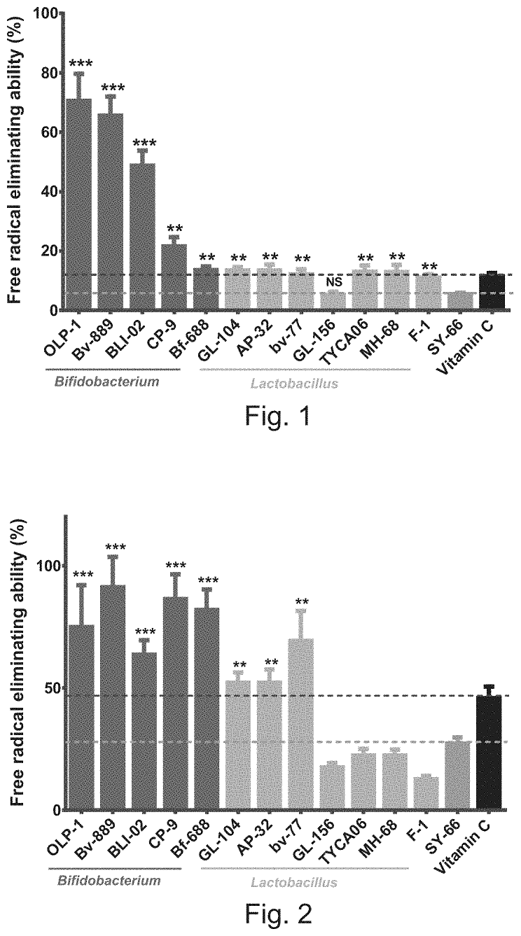

FIG. 1 shows the results of the experiments for determining the free radical eliminating ability of the lactic acid bacterium strains of the present invention (the DPPH assay), wherein *** expresses p<0.005 and ** expresses p<0.01, both indicating a high degree of statistical significance; NS expresses no significant difference. In comparison with the SY-66 strain of Streptococcus thermophiles, the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention have higher free radical eliminating ability.

### Embodiment IV: Analysis of the Free Radical Eliminating Ability of the Fermentation

Metabolites of the Lactic Acid Bacterium Strain

The method to determine the free radical eliminating ability of the fermentation metabolites of the lactic acid bacterium strains is introduced as follows. By a ratio of 1:1, respectively mix 0.2 mM DPPH methanol solutions homogeneously with the following liquids: 1% aqueous solutions of the powders of the fermentation metabolites of the lactic acid bacterium strains of the present invention; 1% aqueous solutions of the powders of the fermentation metabolites of the reference lactic acid bacterium strains, including a GL-156 strain of Lactobacillus paracasei, a TYCA06 strain of Lactobacillus acidophilus, an MH-68 strain of Lactobacillus johnsonii and an F-1 strain of Lactobacillus rhamnosus; a 8.5 μg/ml Vitamin C solution (used as a positive control group); a liquid containing an SY-66 strain (free of anti-oxidant activity) of Streptococcus thermophiles (used as a negative control group); and double-distilled water (used as a blank group). Next, let the mixture liquids react in the dark at an ambient temperature for 30 minutes. Next, the mixture liquids are centrifuged at a speed of 12000 rpm at a temperature of 4° C. for 2 minutes. Next, take 200 μl of liquid from the mixture liquids to a 96-well plate, and measure the values of OD₅₁₇ thereof. The equation for calculating the free radical eliminating ability is the same as stated above.

FIG. 2 shows the results of the experiments for determining the free radical eliminating ability of the fermentation metabolites (0.5%) of the lactic acid bacterium strains of the present invention (the DPPH assay), wherein *** expresses p<0.005 and ** expresses p<0.01, both indicating a high degree of statistical significance. In comparison with the fermentation metabolites of the SY-66 strain of Streptococcus thermophiles, the fermentation metabolites of the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention have higher free radical eliminating ability.

### Embodiment V: Analysis of the Reducing Ability of the Lactic Acid Bacterium Strains of the Present Invention

The reducing ability of an anti-oxidant is usually tested with a FRAP (Ferric-Reducing Ability of Plasma) assay, wherein the integral reducing ability of a sample is regarded as the anti-oxidation ability. In an acidic environment (the pH value thereof is below 3.6), the ferric iron (trivalent iron Fe³⁺) in the FRAP reagent will be reduced into ferrous iron (divalent iron Fe²⁺) by an anti-oxidant, such as Vitamin C, and the color thereof is thus changed, wherein the pigmentation characteristic of TPTZ (2,4,6-Tri-(2-pyridyl)-5-triazine) is used to determine the reducing ability of the sample. While the Fe³⁺-TPTZ complex is reduced into the Fe²⁺-TPTZ complex, the color is changed from yellow to blue. The deeper the blue color, the higher the anti-oxidation ability. Therefore, the OD₅₉₃ value can be used to work out the reducing ability of an anti-oxidant, i.e. the anti-oxidation ability.

In this experiment, let Fe³⁺-TPTZ complex react with the following liquids: the suspensions of the lactic acid bacterium strains of the present invention at a concentration of about 2×10⁹ CFU; the suspensions of the reference lactic acid bacterium strains, including a GL-156 strain of Lactobacillus paracasei, a TYCA06 strain of Lactobacillus acidophilus, an MH-68 strain of Lactobacillus johnsonii and an F-1 strain of Lactobacillus rhamnosus at a concentration of about 2×10⁹ CFU; a 1.25 μg/ml Vitamin C solution (used as a positive control group); a suspension of an SY-66 strain of Streptococcus thermophiles at a concentration of about 2×10⁹ CFU (used as a negative control group). Then, the OD₅₉₃ values thereof are detected. A standard liquid containing a given concentration of FeSO₄ is mixed with the FRAP reagent to obtain a standard calibration curve. The detected OD₅₉₃ values are compared with the standard calibration curve. The equation of the standard calibration curve is used to work out the reducing ability (m/ml, Fe²⁺) of the suspensions of the lactic acid bacterium strains.

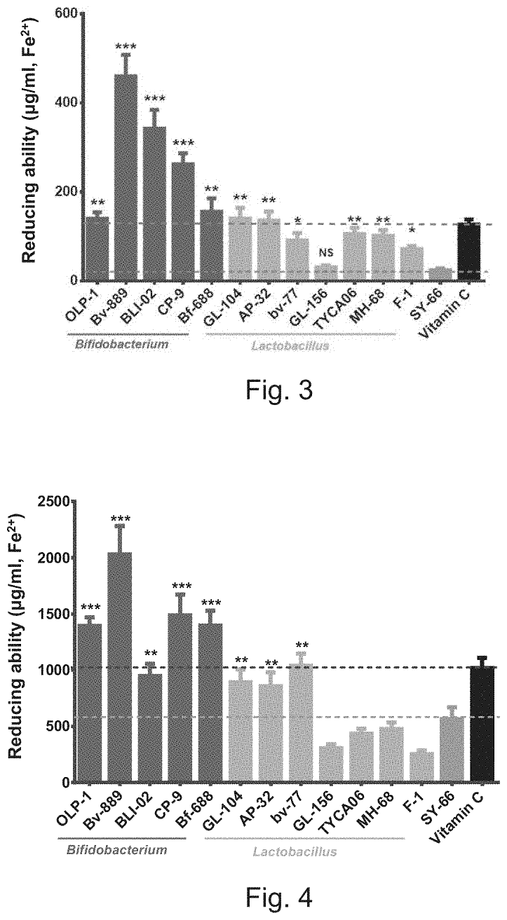

FIG. 3 shows the results of the experiments to determine the reduction ability of the lactic acid bacterium strains of the present invention (the FRAP assay), wherein *** expresses p<0.005 and ** expresses p<0.01, both indicating a high degree of statistical significance, and wherein * expresses that p<0.05, indicating statistical significance; NS expresses no significant difference. In comparison with the SY-66 strain of Streptococcus thermophiles, the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention have a much stronger reducing ability.

### Embodiment VI: Analysis of the Reducing Ability of the Fermentation Metabolites of the Lactic Acid Bacterium Strains of the Present Invention

In this experiment, let Fe³⁺-TPTZ complex react with the following liquids: 0.5% aqueous solutions of the powders of the fermentation metabolites of the lactic acid bacterium strains of the present invention; 0.5% aqueous solutions of the powders of the fermentation metabolites of the reference lactic acid bacterium strains, including a GL-156 strain of Lactobacillus paracasei, a TYCA06 strain of Lactobacillus acidophilus, an MR-68 strain of Lactobacillus johnsonii and an F-1 strain of Lactobacillus rhamnosus; a 5 μg/ml Vitamin C solution (used as a positive control group); a 0.5% aqueous solution of the powder of the fermentation metabolite of an SY-66 strain of Streptococcus thermophiles (used as a negative control group). Then, the OD₅₉₃ values thereof are detected. A standard liquid containing a given concentration of FeSO₄ is mixed with the FRAP reagent to obtain a standard calibration curve. The detected OD₅₉₃ values are compared with the standard calibration curve. The equation of the standard calibration curve is used to work out the reducing ability (μg/ml, Fe²⁺) of the fermentation metabolites of the lactic acid bacterium strains.

FIG. 4 shows the results of the experiments to determine the reduction ability of the fermentation metabolites of the lactic acid bacterium strains of the present invention (the FRAP assay), wherein *** expressesp<0.005 and ** expresses p<0.01, both indicating a high degree of statistical significance. In comparison with the fermentation metabolite of the SY-66 strain of Streptococcus thermophiles, the fermentation metabolites of the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention have a much stronger reducing ability.

### Embodiment VII: Analysis of the Ability of the Lactic Acid Bacterium Strains of the Present Invention to Induce the Intestinal Epithelial Cells to Express Anti-Oxidation Enzymes

The human body has a mechanism to regulate too high oxidative stress. While the oxidative stress is too high, the body will generate antioxidants to deal with such a condition.

For example, the body will synthesize glutathione, ubiquinol and uric acid to absorb free electrons. Besides, the antioxidants absorbed from food, such as Vitamin C and Vitamin E, can also inhibit generation of free radicals. Further, the human body has an anti-oxidation system, i.e. the anti-oxidation enzyme system. Superoxide dismutases (SOD) are important anti-oxidation enzymes, able to convert superoxide into oxygen and hydrogen peroxide through disproportionation reactions. The human body has three kinds of superoxide dismutases, which respectively appear in the external of the cells, in the cytoplasm, and in the mitochondria. There is also an enzyme, called the catalase, able to convert the hydrogen peroxide, which is generated by the superoxide dismutase, into oxygen and water. In the saturation state, a catalase molecule can convert forty-million hydrogen peroxide molecules into oxygen and water.

The Caco-2 cells are the epithelial cells of human colon gland cancer. The structure and function of the Caco-2 cell is similar to that of the differentiated intestinal epithelial cell. The Caco-2 cells have microvilli and the enzyme system related to the epithelial cells of the intestinal brush border. Therefore, the Caco-2 cells are extensively used to simulate the in-vivo physiological activities of intestinal cells. In a cell culture system, the Caco-2 cells may grow into a single layer of cells, which are arranged closely, and which not only morphologically resemble intestinal epithelial cells but also have the same endocytosis phenomenon and the tight-junction structure.

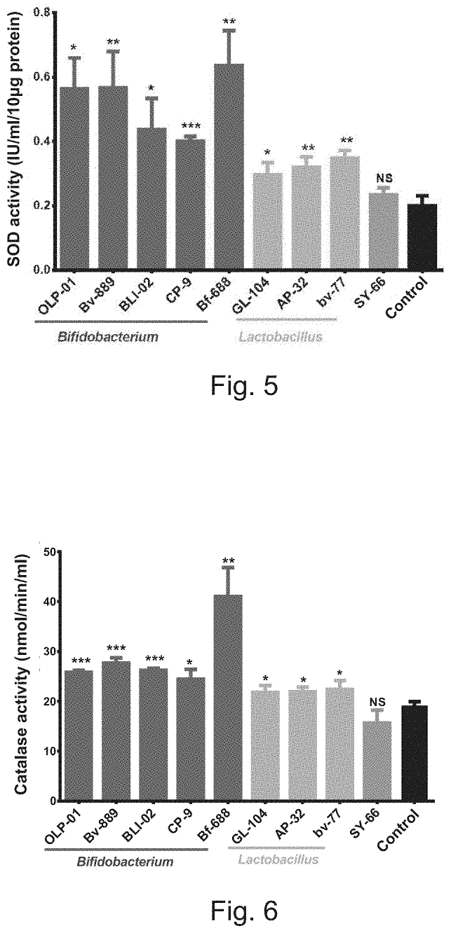

In this experiment, the live strains of the lactic acid bacteria of the present invention (the experiment group), the SY-66 strain of Streptococcus thermophiles (the control group), and a culture solution free of any strain (the control group) are added to the Caco-2 cell culture system by a ratio of cells:probiotics=1:100. The live bacterium strains and the cells are co-cultured for 16 hours. Next, wash out the lactic acid bacterium strains. Next, break the Caco-2 cells and extract the protein to detect the activity of superoxide dismutase (SOD) (the experimental results are shown in FIG. 5) and the activity of catalase (the experimental results are shown in FIG. 6). The experiments respectively adopt the SOD Assay Kit (Cayman Cat. 706002) and the Catalase Assay Kit (Cayman Cat. 707002) in the SOD activity analysis and the catalase activity analysis. The experimental processes are undertaken according to the proposals in the manuals of the kits.

Refer to FIG. 5 and FIG. 6 for the effects of the lactic acid bacterium strains of the present invention to induce the intestinal epithelial cells to express anti-oxidation enzymes, wherein *** expresses p<0.005 and ** expresses p<0.01, both indicating a high degree of statistical significance, and wherein * expresses that p<0.05, indicating statistical significance; NS expresses no significant difference. In comparison with the SY-66 strain of Streptococcus thermophiles, the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention can more efficiently induce the Caco-2 cells to express anti-oxidation enzymes (SOD and catalase) to decompose excessive free radicals in the body and achieve an anti-oxidant effect.

### Embodiment VIII: Analysis of the Ability of the Fermentation Metabolites of the Lactic Acid Bacterium Strains of the Present Invention to Induce the Intestinal Epithelial Cells to Express Anti-Oxidation Enzymes

In this experiment, the powders of the fermentation metabolites of the lactic acid bacterium strains and the SY-66 strain of Streptococcus thermophiles are prepared to form 10% aqueous solutions thereof for determining the ability of the fermentation metabolites of the lactic acid bacterium strains of the present invention to induce the intestinal epithelial cells to generate superoxide dismutase (SOD). The culture medium where no lactic acid bacterium ferments is used as a control group. The experiments adopt the SOD Assay Kit (Cayman Cat. 706002) in the SOD activity analysis. The experimental processes are undertaken according to the proposals in the manual of the kit.

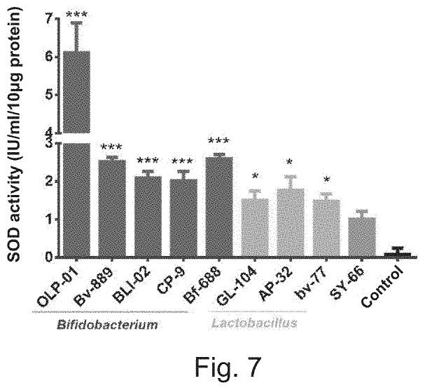

Refer to FIG. 7 for the effects of the fermentation metabolites of the lactic acid bacterium strains of the present invention to induce the intestinal epithelial cells to express anti-oxidation enzymes, wherein *** expresses p<0.005, indicating a high degree of statistical significance, and wherein * expresses that p<0.05, indicating statistical significance. In comparison with the fermentation metabolite of the SY-66 strain of Streptococcus thermophiles, the fermentation metabolites of the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention can more efficiently induce the Caco-2 cells to generate SOD to decompose excessive free radicals in the body and achieve an anti-oxidant effect.

It should be noted: The functionality of lactic acid bacteria to health is not based on the species of bacteria but dependent on the specificities of strains. The strains favorable to health are called the probiotics (Guidelines for the evaluation of probiotics in food; Report of joint FAO/WHO working group on drafting guidelines for the evaluation of probiotics in food; London Ontario, Canada April 30 and May 1, 2002:1-7). According to a paper published in Scientific Reports 2018 (PMID: 30013208), under an aerobic environment, the wild strains of Bifidobacterium longum subsp. Infantis will generate excessive active oxygen radicals (H₂O₂) inside the bacterium bodies because of the action of nicotinamide adenine dinucleotide phosphate (NADPH). Therefore, the wild strains of Bifidobacterium longum subsp. Infantis seem unable to effectively eliminate free radicals but likely to cause death of bacterium bodies or inhibit their growth. The BLI-02 strain of Bifidobacterium longum subsp. Infantis of the present invention has the function of eliminating free radicals. Therefore, the specificity of the BLI-02 strain of Bifidobacterium longum subsp. Infantis of the present invention is different from that of the wild strains of Bifidobacterium longum subsp. Infantis. About the expression of superoxide dismutase (SOD), the paper of a research of National Chung Hsing University (http://hdl.handle.net/11455/50980) found that the B6 strain and 15708 strain of Bifidobacterium longum do not have the activity to induce SOD. Therefore, it is learned from the abovementioned two researches: the BLI-02 strain of Bifidobacterium longum subsp. Infantis and the OLP-01 strain of Bifidobacterium longum subsp. longum are specific strains having an anti-oxidant activity.

In a research by Jayamanne V. S. and Adams M. R. (PMID: 16478503), a strain of Bifidobacterium longum (NCTC11818), a strain of Bifidobacterium breve (NCIMB702258), a strain of Bifidobacterium longum biotype Infantis (NCIMB702205), a strain of Bifidobacterium adolescentis (NCIMB702204) and a strain of Bifidobacterium bifidum (NCIMB702203) are reacted with oxidizing free radicals of H₂O₂. It was found: none of the abovementioned strains has an anti-oxidant activity. In comparison with the strains used in the research of Jayamanne V. S. and Adams M. R., the Bf-688 strain of Bifidobacterium bifidum and the Bv-889 strain of Bifidobacterium breve of the present invention are specific strains having an anti-oxidant activity. In a research by Oberg T. S. et al. (PMID: 23772066), a BL-04 strain and a DSM 10140 strain both belonging to Bifidobacterium animalis subsp. lactis respectively have different levels of anti-oxidant ability. It is rational to infer that not all the strains of Bifidobacterium animalis subsp. lactis have appropriate anti-oxidant activity. Therefore, the CP-9 of Bifidobacterium animalis subsp. lactis is a specific strain having an anti-oxidant activity.

In a research by Chooruk A et al. (PMID: 284748513), the wild L. salivarius and L. rhamnosu, which are directly sourced from oral cavities, do not acquire a satisfied activity of superoxide dismutase (SOD) (only about 0.1-0.2 U). However, the AP-32 strain of Lactobacillus salivarius subsp. salicinius and the bv-77 strain of Lactobacillus rhamnosus of the present invention can achieve a SOD activity of about 1.5-1.75 U. It indicates that the AP-32 strain of Lactobacillus salivarius subsp. salicinius and the bv-77 strain of Lactobacillus rhamnosus of the present invention are specific strains having an anti-oxidant effect. In a research by Narciza 0 et al. (PMID: 30807829), L. reuteri cannot acquire an anti-oxidant function unless it is induced by resveratrol to express the dhaT gene. However, the GL-104 strain of Lactobacillus reuteri of the present invention can directly have an anti-oxidant effect. It indicates that the GL-104 strain of Lactobacillus reuteri of the present invention is a specific strain having an anti-oxidant activity.

In conclusion, compared with the other tested lactic acid bacterium strains, the OLP-01 strain of Bifidobacterium longum subsp. longum; the Bv-889 strain of Bifidobacterium breve; the BLI-02 strain of Bifidobacterium longum subsp. Infantis; the CP-9 strain of Bifidobacterium animalis subsp. lactis; the Bf-688 strain of Bifidobacterium bifidum; the GL-104 strain of Lactobacillus reuteri; the AP-32 strain of Lactobacillus salivarius subsp. salicinius; and the bv-77 strain of Lactobacillus rhamnosus of the present invention have better free radical eliminating ability and better reducing ability and can induce Caco-2 cells to generate more anti-oxidation enzymes. Therefore, the lactic acid bacterium strains of the present invention and the fermentation metabolites thereof have an anti-oxidant activity, able to reduce the concentration of free radicals and inhibit or delay aging of organs.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the appended claims.

### Deposition of Biological Material for Patent Purposes

1. The OLP-01 strain of the present invention
   Deposition Date: Mar. 18, 2019
   Deposition Authority: China General Microbiological Culture Collection Center (CGMCC)
   Address of Deposition Authority: Institute of Microbiology, Chinese Academy of Sciences, NO. 1 West Beichen Road, Chaoyang District, Beijing 100101, China
   Deposition Number: CGMCC No. 17345
   Taxonomic Name: Bifidobacterium longum subsp. longum
2. The Bv-889 strain of the present invention
   Deposition Date: Jul. 23, 2018
   Deposition Authority: China General Microbiological Culture Collection Center (CGMCC)
   Address of Deposition Authority: Institute of Microbiology, Chinese Academy of Sciences, NO. 1 West Beichen Road, Chaoyang District, Beijing 100101, China
   Deposition Number: CGMCC No. 16145
   Taxonomic Name: Bifidobacterium breve
3. The BLI-02 strain of the present invention
   Deposition Date: Jan. 15, 2018
   Deposition Authority: China General Microbiological Culture Collection Center (CGMCC)
   Address of Deposition Authority: Institute of Microbiology, Chinese Academy of Sciences, NO. 1 West Beichen Road, Chaoyang District, Beijing 100101, China
   Deposition Number: CGMCC No. 15212
   Taxonomic Name: Bifidobacterium longum subsp. infantis
4. The CP-9 strain of the present invention
   Deposition Date: Nov. 24, 2014
   Deposition Authority: China Center for Type Culture Collection (CCTCC)
   Address of Deposition Authority: Wuhan University, Wuhan 430072, China
   Deposition Number: CCTCC NO: M2014588
   Taxonomic Name: Bifidobacterium animalis subsp. lactis
5. The Bf-688 strain of the present invention
   Deposition Date: Jun. 18, 2019
   Deposition Authority: China General Microbiological Culture Collection Center (CGMCC)
   Address of Deposition Authority: Institute of Microbiology, Chinese Academy of Sciences, NO. 1 West Beichen Road, Chaoyang District, Beijing 100101, China
   Deposition Number: CGMCC No. 17953
   Taxonomic Name: Bifidobacterium bifidum
6. The GL-104 strain of the present invention
   Deposition Date: Aug. 7, 2009
   Deposition Authority: China Center for Type Culture Collection (CCTCC)
   Address of Deposition Authority: Wuhan University, Wuhan 430072, China
   Deposition Number: CCTCC NO: M209138
   Taxonomic Name: Lactobacillus reuteri
7. The AP-32 strain of the present invention
   Deposition Date: Apr. 10, 2011
   Deposition Authority: China Center for Type Culture Collection (CCTCC)
   Address of Deposition Authority: Wuhan University, Wuhan 430072, China
   Deposition Number: CCTCC NO: M2011127
   Taxonomic Name: Lactobacillus salivarius subsp. salicinius
8. The bv-77 strain of the present invention
   Deposition Date: Nov. 24, 2014
   Deposition Authority: China Center for Type Culture Collection (CCTCC)
   Address of Deposition Authority: Wuhan University, Wuhan 430072, China
   Deposition Number: CCTCC NO: M2014589
   Taxonomic Name: Lactobacillus rhamnosus

What is claimed is:

1. An anti-oxidant composition with a lactic acid bacterium strain or a fermentation metabolite thereof, comprising:
an isolated lactic acid bacterium strain having an anti-oxidant activity or a fermentation metabolite thereof, wherein the lactic acid bacterium strain comprises at least one of an OLP-01 strain of Bifidobacterium longum subsp. longum with a deposition number of CGMCC No. 17345; a Bv-889 strain of Bifidobacterium breve with a deposition number of CGMCC No. 16145; a BLI-02 strain of Bifidobacterium longum subsp. infantis with a deposition number of CGMCC No. 15212; a CP-9 strain of Bifidobacterium animalis subsp. lactis with a deposition number of CCTCC NO: M2014588; a Bf-688 strain of Bifidobacterium bifidum with a deposition number of CGMCC No. 17953; a GL-104 strain of Lactobacillus reuteri with a deposition number of CCTCC NO: M209138; an AP-32 strain of Lactobacillus salivarius subsp. salicinius with a deposition number of CCTCC NO: M2011127; and a bv-77 strain of Lactobacillus rhamnosus with a deposition number of CCTCC NO: M2014589; the abovementioned strains are respectively deposited in China General Microbiological Culture Collection Center (CGMCC) and China Center for Type Culture Collection (CCTCC); and
an excipient, diluent or carrier.

2. The anti-oxidant composition with a lactic acid bacterium strain or a fermentation metabolite thereof according to claim 1, wherein the lactic acid bacterium strains are active strains.

3. The anti-oxidant composition with a lactic acid bacterium strain or a fermentation metabolite thereof according to claim 1, wherein the fermentation metabolite comprises inactivated strains, or a supernatant of a fermentate liquid, a fermentate whey or a dried powder thereof in which bacteria are removed.

4. The anti-oxidant composition with a lactic acid bacterium strain or a fermentation metabolite thereof according to claim 1, wherein the excipient, diluent or carrier is a physiologically-acceptable or pharmaceutically-acceptable excipient, diluent or carrier.

5. The anti-oxidant composition with a lactic acid bacterium strain or a fermentation metabolite thereof according to claim 1, wherein the excipient, diluent or carrier is a food.

6. The anti-oxidant composition with a lactic acid bacterium strain or a fermentation metabolite thereof according to claim 1, which is a pharmaceutical composition and in form of an oral dosage or a topical dosage.

7. The anti-oxidant composition with a lactic acid bacterium strain or a fermentation metabolite thereof according to claim 1, wherein the excipient, diluent or carrier is a cosmeceutically-acceptable excipient, diluent or carrier.

8. The anti-oxidant composition with a lactic acid bacterium strain or a fermentation metabolite thereof according to claim 1, which is a liquid cosmetics, emulsion cosmetics, cream cosmetics, powder cosmetics, block cosmetics or stick cosmetics.

9. A use of a composition with a lactic acid bacterium strain or a fermentation metabolite thereof for anti-oxidation comprising administering to a subject the composition, wherein the composition with a lactic acid bacterium strain or a fermentation metabolite thereof comprises:
an isolated lactic acid bacterium strain having an anti-oxidant activity or a fermentation metabolite thereof, wherein the lactic acid bacterium strain comprises at least one of an OLP-01 strain of Bifidobacterium longum subsp. longum with a deposition number of CGMCC No. 17345; a Bv-889 strain of Bifidobacterium breve with a deposition number of CGMCC No. 16145; a BLI-02 strain of Bifidobacterium longum subsp. infantis with a deposition number of CGMCC No. 15212; a CP-9 strain of Bifidobacterium animalis subsp. lactis with a deposition number of CCTCC NO: M2014588; a Bf-688 strain of Bifidobacterium bifidum with a deposition number of CGMCC No. 17953; a GL-104 strain of Lactobacillus reuteri with a deposition number of CCTCC NO: M209138; an AP-32 strain of Lactobacillus salivarius subsp. salicinius with a deposition number of CCTCC NO: M2011127; and a bv-77 strain of Lactobacillus rhamnosus with a deposition number of CCTCC NO: M2014589; the abovementioned strains are respectively deposited in China General Microbiological Culture Collection Center (CGMCC) and China Center for Type Culture Collection (CCTCC); and
an excipient, diluent or carrier.

10. The use of the composition with the lactic acid bacterium strain or the fermentation metabolite thereof for anti-oxidation according to claim 9, wherein the lactic acid bacterium strains are active strains.

11. The use of the composition with the lactic acid bacterium strain or the fermentation metabolite thereof for anti-oxidation according to claim 9, wherein the fermentation metabolite comprises inactivated strains, or a supernatant of a fermentate liquid, a fermentate whey or a dried powder thereof in which bacteria are removed.

12. The use of the composition with the lactic acid bacterium strain or the fermentation metabolite thereof for anti-oxidation according to claim 9, wherein the excipient, diluent or carrier is a physiologically-acceptable or pharmaceutically-acceptable excipient, diluent or carrier.

13. The use of the composition with the lactic acid bacterium strain or the fermentation metabolite thereof for anti-oxidation according to claim 9, wherein the excipient, diluent or carrier is a food.

14. The use of the composition with the lactic acid bacterium strain or the fermentation metabolite thereof for anti-oxidation according to claim 9, wherein the composition with the lactic acid bacterium strain or the fermentation metabolite thereof is a pharmaceutical composition and in form of an oral dosage or a topical dosage.

15. The use of the composition with the lactic acid bacterium strain or the fermentation metabolite thereof for anti-oxidation according to claim 9, wherein the excipient, diluent or carrier is a cosmeceutically-acceptable excipient, diluent or carrier.

16. The use of the composition with the lactic acid bacterium strain or the fermentation metabolite thereof for anti-oxidation according to claim 9, wherein the composition with the lactic acid bacterium strain or the fermentation metabolite thereof is a liquid cosmetics, emulsion cosmetics, cream cosmetics, powder cosmetics, block cosmetics or stick cosmetics.
